(19) United States
(12) Patent Application Publication
(10) Pub. No.: US 2023/0365859 A1
(43) Pub. Date: Nov. 16, 2023

(54) NOVEL ELECTROCHEMILUMINESCENCE CO-REACTANT, ELECTROLYTE SOLUTION AND ELECTROCHEMILUMINESCENCE SYSTEM COMPRISING SAME

(71) Applicant: Elips Diagnostics Inc., Seoul (KR)

(72) Inventor: Ik-Soo Shin, Seoul (KR)

(21) Appl. No.: 18/224,121

(22) Filed: Jul. 20, 2023

Related U.S. Application Data
(63) Continuation of application No. PCT/KR2022/002654, filed on Feb. 23, 2022.

(30) Foreign Application Priority Data
Feb. 26, 2021 (KR) 10-2021-0026210
Feb. 8, 2022 (KR) 10-2022-0016178

(51) Int. Cl. C09K 11/07; G01N 33/58; G01N 21/64

(52) U.S. Cl. CPC C09K 11/07; G01N 33/582; G01N 21/6486; C09K 2211/1029

(57) ABSTRACT

Provided herein, inter alia, an electrochemiluminescence (ECL) co-reactant for an electrochemiluminescence system; an electrolyte composition (e.g., electrolyte solution) including the same; an electrochemiluminescence system for detecting a biological material using the same.

### CROSS-REFERENCES TO RELATED APPLICATIONS

This application is a continuation of International Patent Application PCT/KR2022/002654 filed Feb. 23, 2022, which claims the benefit of priority of Korean Patent Application Nos. 10-2021-0026210 filed on Feb. 26, 2021 and 10-2022-0016178 filed on Feb. 8, 2022, which are incorporated herein by reference in its entirety and for all purposes.

### TECHNICAL FIELD

Provided herein, inter alia, an electrochemiluminescence (ECL) co-reactant for an electrochemiluminescence system; an electrolyte composition (e.g., electrolyte solution) including the same; an electrochemiluminescence system for detecting a biological material using the same. The electrochemiluminescence co-reactant may include a compound (or “pyridine derivative”) having a formula of Chemical Formula (I) as described herein, or a pharmaceutically acceptable salt thereof. The electrochemiluminescence co-reactant can react with an electrochemiluminescent label or a luminescent species such as a polycyclic aromatic hydrocarbon compound, a metal complex compound, a quantum dot, or nanoparticles, and the like and provide substantially enhanced detection signal during measuring electrochemiluminescence. The electrochemiluminescence co-reactant can be widely and generally applied to various immunoassay methods, chemical analysis methods and diagnostic devices while the voltage application condition can be improved in application of such methods.

### BACKGROUND

Research has been conducted for rapid, highly specific, sensitive and accurate methods of detecting and quantifying chemical, biochemical and biological materials. Improvements in analytical performance, such as sensitivity, may be important for sensing a substantially small amount of a specific analyte in biological samples.

One approach to improving analytical sensitivity is to take advantage of methods available for highly sensitive photodetection (e.g., photomultiplier tubes). In the related art, luminescent indicator molecules has been used as important improving factor. For example, a luminescent label associated with an analyte of interest (or a binding factor for the analyte of interest) has been used to quantitatively detect the presence of the analyte (or a binding partner).

The amount of analyte can be quantitatively determined when the analyte participates in a reaction that induces the regulation of luminescence as described below. For example, i) an analyte may react with another species to regulate the luminescent properties of the second species, ii) the analyte may undergo chemical modifications that regulate the luminescent properties of the analyte itself, iii) the analyte may be a catalyst (for example, an enzyme) that induces the reactions of other species, or iv) the analyte may participate in a reaction that produces a species, and then participate in subsequent reactions that induce luminescence regulation.

Methods for detecting luminescent indicator molecules include photoluminescence, chemiluminescence, and electrochemiluminescence (ECL). Among various light detection systems, with an electrochemiluminescence (ECL) system, it is possible to construct an inexpensive and compact diagnostic system and minimize signal interference caused by background signals from other interferers because a light source, which has a large volume and is expensive, is not required.

For example, electrochemiluminescence (ECL) is an electrical light generation phenomenon discovered in about 1960, and the electrochemiluminescence (ECL) i) induces an oxidation reaction of a specific luminescence material through the application of voltage, ii) allows an intermediate reactant produced thereafter to change into a final product in an excited state through a secondary chemical reaction, and then iii) generates light while the excited state is converted to the ground state. Such ECL has been used as a detection method in high-end medical immunodiagnostic devices.

As a form of utilization, a COBAS® diagnostic device using the ELECSYS® assay from Roche and a Mesoscale Discovery ECL Systems series are the only immunodiagnostic devices based on electrochemiluminescence (ECL), and dominate the high-end immunodiagnostic device market around the world. These methods generate ECL using a ruthenium compound e.g., [Ru(bpy)₃²⁺] as a luminophore (i.e. “electrochemiluminescent labelling compound” or “electrochemiluminescent label”) and tripropylamine (TPrA) as a co-reactant. However, TPrA may have a disadvantage, for example, because tripropylamine is hydrophobic, the luminescence efficiency is not sufficient under platinum and gold electrode conditions. Furthermore, in the 60-year history of electrochemiluminescence development, there has not been a co-reactant that exhibits greater luminescence efficiency than tripropylamine.

Tripropylamine (TPrA) has been generally used as a co-reactant because it allows efficient electrochemiluminescence (ECL) in organic solvent system as well as aqueous media and even at physiological pH 7.4. However, TPrA also has a disadvantage in that it has volatile toxicity and must be used at high concentrations (generally up to 180 mM) to obtain high electrochemiluminescence (ECL) signals. Further, TPrA has the inconvenience of requiring a highly concentrated buffer solution to prepare a solution because it has a slow electrochemical oxidation rate, limits electrochemiluminescence (ECL) efficiency, and is basic. In addition, TPrA has a disadvantage in that the deviation between the same detection signals is relatively large, and it chemically reacts with carbon dioxide in air.

Furthermore, the electrochemiluminescence (ECL) efficiency of ruthenium pyridine (e.g., [Ru(bpy)₃²⁺]) and tripropylamine (TPrA) may depend on electrode materials. For example, platinum (Pt) and gold (Au) electrodes can be covered with an oxide layer of an positive electrode at the surface where electrochemiluminescence occurred, which can suppress the direct oxidation of tripropylamine, resulting in an decrease in electrochemiluminescence intensity. In contrast, a polished glassy carbon (GC) electrode has a relatively fast electrochemical oxidation rate of tripropylamine, which results in a greater amount of tripropylamine oxidized on the electrode surface and a substantially greater emission intensity.

Therefore, there is a demand for a new electrochemiluminescence co-reactant capable of improving the emission intensity and detection reproducibility of electrochemiluminescence (ECL) systems.

### SUMMARY

Provided herein is, inter alia, an electrochemiluminescence (ECL) system (the “system”) and components thereof (e.g., electrolyte solution) that can be widely applicable to testing blood sugar, cholesterol sensors, molecular diagnostics, or antibody detection by immunoassay. In preferred aspect, a pyridine derivative may be used as co-reactant with ruthenium pyridine, such that the luminescence intensity may be enhanced in an electrochemiluminescence system and a fast reaction may occur. For example, in the system using the pyridine derivative co-reactant, electrode materials in the system may not be affected and luminescence efficiency may be improved compared to the system using a conventional co-reactant, e.g., tripropylamine.

In an aspect, provided is an electrochemiluminescence (ECL) co-reactant including a pyridine derivative compound (the “compound”) having a structure of Chemical Formula (I):

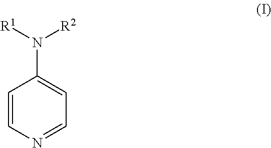

wherein R¹ and R² are the same as or different from each other, and each R¹ and R² is independently a hydrogen, a halogen, a C₁-C₆ straight-chain alkyl group, a C₁-C₆ branched-chain alkyl group, or C₃-C₆ cyclic alkyl group, a C₁-C₆ alkoxy group; and a C₁-C₆ haloalkyl group, or a pharmaceutically acceptable salt thereof.

In an aspect, provided is an electrolyte solution including an electrochemiluminescent label; an electrochemiluminescence co-reactant comprising a compound having a structure of Chemical Formula (I) as described herein, or a pharmaceutically acceptable salt thereof.

In an aspect, provided is an electrochemiluminescence system including an electrochemical cell; the electrolyte solution as described herein; and a photodetector connected to the electrochemical cell. The electrolyte solution may be filled in the electrochemical cell.

In an aspect, provided is a kit for electrochemiluminescence immunoassay or molecular diagnosis. The kit includes the electrolyte solution as described herein.

In an aspect, provided is a method of detecting a biological material in a sample. The method includes steps of: (a) placing an admixture comprising the electrolyte solution as described herein and a sample into an electrochemical cell; and (b) measuring an electrochemiluminescence intensity (ECL intensity) from the admixture while varying an input voltage.

According various exemplary embodiments, the pyridine derivative as described herein (e.g., compounds of Chemical Formula (I)) can be widely applied in various biochemical or electrochemiluminescent assays such as immunoassays, which will be benefited by excellent handleability as a solid compound, improved potential (or voltage) conditions for luminescence, and improved luminescence efficiency even used at a low concentration.

Other aspects of the invention are disclosed infra.

### BRIEF DESCRIPTION OF DRAWINGS

FIG. 1 shows an exemplary electrochemiluminescence system including a potentiostat and a photomultiplier tube according to an exemplary embodiment of the disclosure.

FIG. 2 shows the results of measuring a cyclic voltammogram (CV) in an electrochemiluminescence system including 5 mM co-reactant and 1 μM [Ru(bpy)₃]²⁺ ruthenium pyridine in 1×PBS (pH 7.4)(Scan rate: 0.1 V/s, WE: GC, CE: Pt, RE: Ag/AgCl).

FIG. 3 shows the results of measuring ECL intensity in an electrochemiluminescence system including 5 mM co-reactant and 1 μM [Ru(bpy)₃]²⁺ ruthenium pyridine in 1×PBS (pH 7.4) (Scan rate: 0.1 V/s, WE: GC, CE: Pt, RE: Ag/AgCl).

FIG. 4 shows the results of measuring the cyclic voltammogram (CV) in an electrochemiluminescence system including 5 mM co-reactant and 1 μM [Ru(bpy)₃]²⁺ in 1×PBS (pH 7.4) (Scan rate: 0.1 V/s, WE: Pt, CE: Pt, RE: Ag/AgCl).

FIG. 5 shows the results of measuring ECL intensity in an electrochemiluminescence system including 5 mM co-reactant and 1 μM [Ru(bpy)₃]²⁺ in 1×PBS (pH 7.4)(Scan rate: 0.1 V/s, WE: Pt, CE: Pt, RE: Ag/AgCl).

FIG. 6 is a plot showing the dependence of ECL intensity of 1 μM [Ru(bpy)₃]²⁺ ruthenium pyridine on the concentration of 4-DMAP and TPrA (1 to 100 mM) in 1×PBS (pH 7.4). The potential was increased stepwise from 0 V to 1.6 V (WE: Pt, CE: Pt, RE: Ag/AgCl).

FIG. 7 is a plot showing the dependence of ECL intensity of 10 μM [Ru(bpy)₃]²⁺ ruthenium pyridine on the concentration of 4-DMAP and TPrA (1 to 100 mM) in 1×PBS (pH 7.4). The potential was increased stepwise from 0 V to 1.6 V (WE: Pt, CE: Pt, RE: Ag/AgCl).

FIG. 8 shows the results of measuring ECL intensity under various pH conditions using [Ru(bpy)₃]²⁺ ruthenium pyridine (1 μM and 10 μM) (WE: Pt, CE: Pt, RE: Ag/AgCl).

FIGS. 9A-9C are graphs illustrating the comparison of chemiluminescence intensities when 4-DMAP and TPrA are used as co-reactants in an acetonitrile (ACN) solution. Glassy carbon (FIG. 9A), platinum (FIG. 9B) and gold (FIG. 9C) are used as working electrodes (CE: Pt, RE: Ag/AgCl).

FIG. 10 shows graphs illustrating a linear sweep voltammogram and electrochemiluminescence intensity when glassy carbon is used as a working electrode under the circumstances of using 4-DMAP as a co-reactant in an acetonitrile (ACN) solution (CE: Pt, RE: Ag/Ag⁺ (3 M AgNO₃), scan rate: 0.1 V/s).

FIG. 11 shows the results of obtaining the difference in electrochemiluminescence signals generated by changing the concentration of a Ru(bpy)₃²⁺ luminophore when 7 mM 4-DMAP and 7 mM tripropylamine were each used as co-reactants in the form of a calibration curve.

FIG. 12 shows the results of performing electrochemiluminescence immunodiagnosis on the “severe acute respiratory syndrome (SARS-CoV-2) neutralizing antibody (anti-SARS-CoV-2)” present in ten saliva samples inoculated with a vaccine using 4-DMAP or tripropylamine as a co-reactant.

### DETAILED DESCRIPTION

Electrochemiluminescence is a luminescence process which occurs while a compound generated at an electrode undergoes an electron transfer reaction with high energy to form an excited state that emits light. Luminescent labeling reagents used for electrochemiluminescence may include, but not be limited to transition metal complex compounds, luminescent organic semiconductors, quantum dot materials, perovskite nanoparticles, metal nanoparticles or carbon nanoparticles. These organic and inorganic luminescent labels have been used for a wide range of bioanalysis.

The oxidation reaction may be the basic principle of electrochemiluminescence, and may include the loss of electrons at the surface of an electrode by a luminescent substrate and a composition thereof during the reaction process. For example, an electron donor may lose a hydrogen ion (H⁺) to become a strong reducing agent which reduces a luminescent substrate in the excited state, and thereafter, the luminescent substrate may emit photons to return to the ground state. This process may be repeatedly performed at the surface of the electrode, and photons can be generally emitted continuously to keep the substrate concentration constant.

As an example, there is an electrochemiluminescence system of ruthenium pyridine (e.g., Ru(bpy)₃) and tripropylamine (TPrA). The electrochemiluminescence reaction may include a specific chemiluminescence reaction induced by electrochemistry at the surface of the electrode. For example, ruthenium pyridine conjugated to or labeled at antigen-antibody complexes may be excited by electrochemistry in the presence of tripropylamine, and a redox reaction occurs to release photons, which can be sensed by a photomultiplier tube. This process can be repeatedly performed to produce many photons, which thus amplify the optical signal. In general, labels used in electrochemiluminescence assays may bind to antibody or antigen molecules with different chemical structures to produce labeled antibodies or antigens.

In conventional electrochemiluminescence methods, tripropylamine (TPrA) has been used as a co-reactant. Because the TPrA is in a liquid phase, this co-reactant is difficult to handle, and due to slow reactivity of TPrA, high concentrations thereof may be required, and it may be greatly affected by electrode materials. Thus, TPrA may have limited luminescence efficiency the limitations of toxicity and instability.

### Definition

The abbreviations used herein have their conventional meaning within the chemical and biological arts. The chemical structures and formulae set forth herein are constructed according to the standard rules of chemical valency known in the chemical arts.

The term “halogen” or “halo” designates —F, —Cl, —Br or —I.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group (e.g., “alkenyl” or “alkynyl”) is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. In some embodiments, the alkyl moiety may be an alkenyl moiety. In some embodiments, the alkyl moiety may be an alkynyl moiety.

A term “alkoxy” is an alkyl attached to the remainder of the molecule via an oxygen linker (—O—).

The term “cycloalkyl” as used herein, by themselves or in combination with other terms, mean, cyclic versions of “alkyl”. Cycloalkyl is not aromatic. Examples of cycloalkyl include, but are not limited to, cyclopropyl, cyclobutyl, cyclopentyl, cyclohexyl, 1-cyclohexenyl, 3-cyclohexenyl, cycloheptyl, and the like.

In certain embodiments, the alkyl, alkoxy, cycloalkyl as used here may be optionally substituted. The term “substituted”, “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. In certain embodiments, alkyl groups may be optionally substituted or unsubstituted, e.g., with hydroxyl, halogen, amine, acetyl, or other alkyl or alkoxyl, or the like.

As used herein, the definition of each expression, e.g. alkyl, R¹ and R², etc., when it occurs more than once in any structure, is intended to be independent of its definition elsewhere in the same structure.

The term “pharmaceutically-acceptable salts” refers to the relatively non-toxic, inorganic and organic acid addition salts of compounds disclosed herein and inorganic and organic basic addition salts of the compounds disclosed herein. It is understood that the reference to a pyridine derivative includes the neutral compound and any pharmaceutically acceptable salt for of the pyridine derivative. The pharmaceutically acceptable salt forms which may be selected on the basis of a chosen route of administration and according to standard pharmaceutical practice.

As set out above, certain embodiments of the pyridine derivatives may contain a basic functional group, such as amino, and are capable of forming pharmaceutically acceptable salts with pharmaceutically acceptable acids. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds provided in this disclosure may contain one or more acidic functional groups and, thus, are capable of forming pharmaceutically acceptable salts with pharmaceutically acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Certain compounds provided in this disclosure may exist in particular geometric or stereoisomeric forms. The disclosure contemplates all such compounds, including cis- and trans-isomers, R- and S-enantiomers, diastereomers, the racemic mixtures thereof, and other mixtures thereof, as falling within the scope of the invention. Additional asymmetric carbon atoms may be present in a substituent such as an alkyl group. All such isomers, as well as mixtures thereof, are included in this invention.

A term “co-reactant,” as used herein and understood in the art, refers to a chemical species or compound that undergoes an electrochemical reaction (e.g., oxidation or reduction) and helps generating (e.g., enhancing or increasing) luminescence during electrochemical reactions in the presence of chemiluminescent molecules (in other words, “electrochemiluminescent labelling compound” or “electrochemiluminescent label”). In certain aspect, the co-reactant enhances or increases the efficiency of luminescence or chemo luminescence of the chemiluminescent molecules (“electrochemiluminescent labelling compound” or “electrochemiluminescent label”), e.g., via radical reaction to reach excited state.

A term “about” as used herein is understood as within a range of normal tolerance in the art, for example within 2 standard deviations of the mean. For example, “about” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

When a range is described for a variable, it will be understood that the variable includes all values including the end points described within the stated range. For example, the range of “5 to 10” will be understood to include any subranges, such as 6 to 10, 7 to 10, 6 to 9, 7 to 9, and the like, as well as individual values of 5, 6, 7, 8, 9 and 10, and will also be understood to include any value between valid integers within the stated range, such as 5.5, 6.5, 7.5, 5.5 to 8.5, 6.5 to 9, and the like.

### Co-Reactants and Compositions

Thus, provided herein, inter alia, is a co-reactant including a solid compound 4-dimethylaminopyridine (4-DMAP) for electrochemiluminescence. The co-reactant may provide imminent luminescence reaction and have excellent luminescence efficiency.

In an aspect, provided is an electrochemiluminescence (ECL) co-reactant including a compound represented by Chemical Formula (I), or a pharmaceutically acceptable salt thereof.

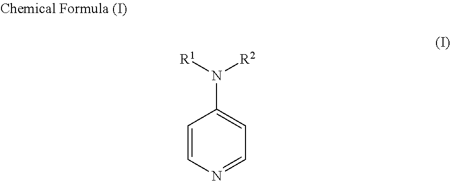

In Chemical Formula (I), wherein R¹ and R² are the same as or different from each other, and each R¹ and R² is independently a hydrogen, a halogen, a C₁-C₆ straight-chain alkyl group, a C₁-C₆ branched-chain alkyl group, or C₃-C₆ cyclic alkyl group, a C₁-C₆ alkoxy group; and a C₁-C₆ haloalkyl group.

In certain embodiments, R¹ and R² may be the same as or different from each other, and may be a C₁-C₄ straight-chain alkyl group, a C₁-C₄ branched alkyl group; or a C₁-C₄ haloalkyl group. In certain embodiments, the compound of Chemical Formula (I) may be 4-dimethylaminopyridine having a structure of

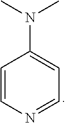

In an aspect, provided is an electrolyte composition that includes the co-reactant including the compound having the Chemical Formula (I) as described herein.

In an aspect, provided is an electrolyte composition that includes (i) an electrochemiluminescent labelling compound and (ii) co-reactant including the compound having the Chemical Formula (I) as described herein.

In certain embodiments, the electrolyte composition may further include a solvent component comprising one or more selected from the group consisting of water, phosphate buffer saline (PBS), acetonitrile (ACN), dichloromethane, ethanol, methanol, tetrahydrofuran (THF), dimethyl sulfoxide (DMSO), dimethylformamide (DMF), ethylene carbonate (EC) and propylene carbonate (PC).

In certain embodiments, the electrolyte composition may be in a form of a liquid electrolyte including a salt, water and/or an organic solvent; a solid electrolyte in which a salt is dissolved in a polymer; a gel-type electrolyte including a polymer, a salt, water, and an organic solvent; or an ionic gel electrolyte including a block copolymer and an ionic liquid, but is not limited thereto. Preferably, the electrolyte composition may be in a form of a solution, i.e., electrolyte solution.

In an aspect, provided is an electrolyte solution that include (i) an electrochemiluminescent labelling compound and (ii) a co-reactant including a compound having the Chemical Formula (I).

In certain embodiments, the electrolyte solution may include a solvent component comprising one or more selected from the group consisting of water, phosphate buffer saline (PBS), acetonitrile (ACN), dichloromethane, ethanol, methanol, tetrahydrofuran (THF), dimethyl sulfoxide (DMSO), dimethylformamide (DMF), ethylene carbonate (EC) and propylene carbonate (PC).

In certain embodiments, the electrolyte solution may have a pH in a range of about 2 to 12, about 3 to 12, about 4 to 12, about 5 to 12, about 6 to 12, about 7 to 12, or about 7 to 11. In certain preferred embodiments, the electrolyte solution may have a pH in a range of about 7.4 to 10.

In certain embodiments, the electrochemiluminescent label in the electrolyte composition or electrolyte solution may include one or more selected from the group consisting of a transition metal complex compound, a luminescent organic semiconductor, a quantum dot material, perovskite nanoparticles, metal nanoparticles and carbon nanoparticles.

The electrochemiluminescent label may suitably include one or more transition metal compounds thereof. For example, the transition metal compounds may include one or more selected from the group consisting of ruthenium (Ru), iridium (Ir), rhenium (Re), platinum (Pt), osmium (Os), copper (Cu), and iron (Fe).

The electrochemiluminescent label may suitably include one or more ionic transition metal complex compounds. Non-limiting examples of the ionic transition metal complex compound may include one or more selected from the group consisting of tris(2,2′-bipyridine)ruthenium(II)bis(hexafluorophosphate) (Ru(bpy)₃(PF₆)₂), tris(4,7-diphenyl-1,10-phenanthroline)ruthenium(II) bis(hexafluorophosphate) (Ru(dp-phen)₃(PF₆)₂), bis(2-phenylpyridine)(2,2′-dipyridine)iridium(III) (hexafluorophosphate) (Ir(ppy)₂(bpy)PF₆), bis(2-phenylpyridinex4,4′-di-tert-butyl-2,2′-dipyridyl)iridium(III) (hexafluorophosphate) (Ir(dtbbpy)(ppy)₂PF₆), 4′-di-tert-butyl-2,2′-dipyridyl-bis[2-(2′,4′-difluorophenyl)pyridine]iridium(III)(hexafluorophosphate) (Ir(ppy-F₂)₂(dtbbpy)PF₆), iridium bis[5-(trifluoromethyl)-2-(4-(trifluoromethyl)phenyl)pyridine] picolinate (Ir(ppy-(CF₃)₂)₂(pico), tris[2-(p-tolyl)pyridine]iridium(III) (Ir(mppy)₃), 1,10-[phenanthroline]rhenium(I)(hexafluorophosphate) (Re(phen)PF₆), platinum(II) coproporphyrin [PtCP] and tris(2,2′-bipyridine)osmium(II)(hexafluorophosphate) (Os(bpy)₃(PF₆)₂).

The electrochemiluminescent label may suitably include one or more luminescent organic semiconductors may include a conjugated organic semiconductor capable of emitting light, such as a luminescent single molecule or a polymer. For example, the luminescent organic semiconductor may include one or more selected from the group consisting of luminol and rubrene and derivatives thereof, anthracene and derivatives thereof, pyrene and derivatives thereof, decycloxyphenyl substituted poly(1,4-phenylene vinylene) (super yellow), poly(2-methoxy-5-(2-ethylhexyloxy)-1,4-phenylenevinylene (MEH-PPV), poly(2-methoxy-5-(3′,7′-dimethyloctyloxy)-1,4-phenylenevinylene) (MEMO-PPV), and poly(9,9-dioctylfluorene-alt-benzothiadiazole (F8BT), but examples are not limited thereto.

The electrochemiluminescent label may suitably include one or more quantum dot materials including an inorganic compound of Group 13-15 or Group 12-15 elements. For example, the quantum dot material including the inorganic compound may include one or more selected from the group consisting of cadmium selenide (CdSe), cadmium sulfide (CdS), zinc selenide (ZnSe), indium phosphide (InP), lead sulfide (PbS) and lead selenide (PbSe), but examples are not limited thereto.

The electrochemiluminescent label may suitably include one or more perovskite nanoparticles including halide-based perovskites. For example, the halide-based perovskite may be represented by a chemical formula of ABX₃, A₂BX₆ or A₃B₂X₉, where A may be an organic or inorganic cation, B may be a metal cation, and X may be a halide anion.

The electrochemiluminescent label may suitably include one or more metal nanoparticles including metal atom clusters having a dimension of 1 nm or less, which exhibit discrete energy levels. The metal nanoparticles may include gold (Au) nanoparticles, silver (Ag), copper (Cu), or silver (Ag)-gold (Au) bimetallic nanoparticles.

The electrochemiluminescent label may suitably include one or more carbon particles such as a graphene quantum dot (GQD) or a carbon quantum dot (CQD), but are not limited thereto.

### Systems, Methods and Kits

Further, in an aspect, provided is an electrochemiluminescence system including: an electrochemical cell and an electrolyte solution as described herein. The electrolyte solution may include the co-reactant as described herein and the electrochemiluminescent label as described herein. The electrochemiluminescence may further include a photodetector connected to the electrochemical cell.

The electrolyte solution may be in a form of a liquid electrolyte including a salt, water and/or an organic solvent; a solid electrolyte in which a salt is dissolved in a polymer; a gel-type electrolyte including a polymer, a salt, water, and an organic solvent; or an ionic gel electrolyte including a block copolymer and an ionic liquid but is not limited thereto. Preferably, the “salt” may include an organic or inorganic ionic compound and may include one or more selected from the group of phosphate, nitrate, sulfate, lithium salt, sodium salt, potassium salt, calcium salt, magnesium salt, and ammonium salt, but is not limited thereto.

In certain embodiments, the electrolyte solution used in the electrochemiluminescence system as described herein may be a solution including water or an organic solvent of one or more selected from the group consisting of phosphate buffer saline (PBS), a Tris buffer solution, acetonitrile (ACN), dichloromethane, ethanol, methanol, tetrahydrofuran (THF), dimethyl sulfoxide (DMSO), dimethylformamide (DMF), ethylene carbonate (EC) and propylene carbonate (PC), but is not limited thereto.

In certain embodiments, the electrolyte solution in the electrochemiluminescence system as described herein may have a pH in a range of about 2 to 12, about 3 to 12, about 4 to 12, about 5 to 12, about 6 to 12, about 7 to 12, or about 7 to 11. In certain preferred embodiments, the electrolyte solution may have a pH in a range of about 7.4 to 10.

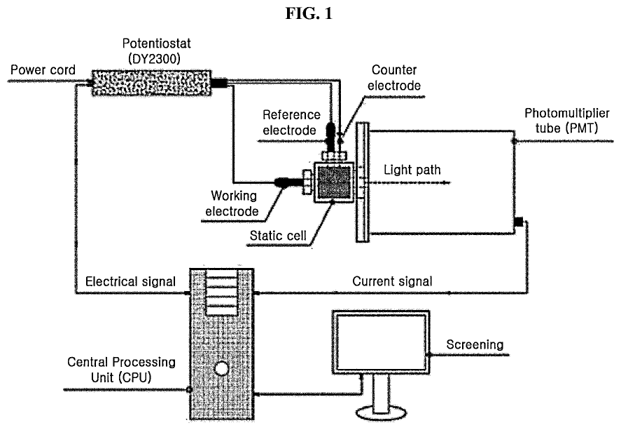

FIG. 1 shows an exemplary electrochemiluminescence system for performing electrochemiluminescence measurements according to an exemplary embodiment of the present invention. The electrochemiluminescence system includes an electrochemical cell including an electrolyte solution, a potentiostat and a photomultiplier tube (PMT). The photomultiplier tube (PMT) is connected to a potentiostat and driven at the same time. Therefore, an electrochemiluminescence reaction may be caused by inducing a reaction in an electrochemical cell using a potentiostat for electrochemiluminescence measurement. For electrochemiluminescence measurement, a potentiostat and electrochemiluminescence measurement software may be run to measure the luminescence intensity of electrochemiluminescence, and the like.

The electrode that constitutes the electrochemical cell may include a working electrode, a reference electrode, a working electrode, and a counter electrode, but is not limited thereto.

The working electrode may be an electrode formed of one or more selected from the group consisting of carbon, platinum (Pt), gold (Au), silver (Ag), nickel (Ni), stainless steel, palladium, tin, indium and silicon elements, but is not necessarily limited thereto.

The counter electrode may be an electrode formed of one or more selected from the group consisting of carbon, platinum (Pt), gold (Au), silver (Ag), nickel (Ni), stainless steel, palladium, tin, indium and silicon elements, but is not necessarily limited thereto.

The reference electrode may include one or more selected from the group consisting of a silver (Ag)-based Ag pseudo-reference electrode, an Ag/AgCl electrode, a Ag/AgNO₃ electrode, a mercury (Hg) calomel electrode, a Hg/HgO electrode, and a Hg₂SO₄ electrode, but is not limited thereto.

In an aspect, provided also is a detection method using an electrochemiluminescence system. In certain aspect, the method is for detecting a biological material in a sample. The method may include: (a) placing an admixture including an electrolyte solution and a sample into an electrochemical cell in the electrochemiluminescence system such that a reaction occurs in the admixture; and (b) measuring the electrochemiluminescence intensity (ECL intensity) from the admixture while varying an input voltage. The method may include using an electrochemiluminescence-based detector for detecting an optical signal.

In the step (a), the sample may be added to the electrolyte solution including a co-reactant and an electrochemiluminescence label (e.g., ruthenium pyridine), and the resulting admixture may react. The sample may include a biological sample (e.g., serum, urine, stool, blood, cell, mucous, discharge, or tissue fluid) obtained from a subject (e.g., human, human patient, or animal), but is not limited thereto.

In certain embodiments, the biological material to be detected from the sample may include, but not be limited to, a peptide, a protein, an antibody, an antigen, a nucleic acid, a virus, a cell, a tissue, a small molecule, an oligosaccharide, a monosaccharide, a lipid, or combinations thereof.

In the step (b), the electrochemiluminescence intensity (ECL intensity) may be measured from the admixture according to the input potential (or voltage), for example, by varying input potential, using an electrochemiluminescence-based photodetector. There are various types of photodetectors which are devices for measuring such luminescence. For example, the photodetector may include a photodiode including silicon, germanium, germanium-phosphide, indium-gallium-arsenide, and lead-sulfide; a photomultiplier tube (PMT); a charge coupled device (CCD); an electron-multiplying charge coupled device (EMCCD); or a scientific complementary metal-oxide-semiconductor (sCMOS), but is not limited thereto.

The method may further include a step of preparing the electrolyte solution as described herein.

In certain embodiments, the method may further include a step of calculating a concentration of the biological material in the sample or determining the presence of the biological material in the sample.

In certain aspect, the method may include an electrochemiluminescence immunoassay detection method through an electrochemical reaction. The method may include steps of combining an electrolyte solution as described herein with a sample containing biological material (e.g., antigen or antibody) for immunoassay; and (b) measuring the resulting electrochemiluminescence intensity (ECL intensity) while varying an input voltage.

In an aspect, provided is a kit for electrochemiluminescence immunoassay or molecular diagnosis, including the electrolyte solution as described herein. The electrolyte solution includes the co-reactant and the electrochemiluminescent label as described herein.

In certain aspect, the electrolyte solution in the kit may further include a solvent component. Particular, the solvent component of the electrolyte solution may include water or an organic solvent that may include one or more selected from the group consisting of phosphate buffer saline (PBS), a Tris buffer solution, acetonitrile (ACN), dichloromethane, ethanol, methanol, tetrahydrofuran (THF), dimethyl sulfoxide (DMSO), dimethylformamide (DMF), ethylene carbonate (EC) and propylene carbonate (PC), but is not limited thereto.

In certain embodiments, the electrolyte solution in the kit as described herein may have a pH in a range of about 2 to 12, about 3 to 12, about 4 to 12, about 5 to 12, about 6 to 12, about 7 to 12, or about 7 to 11. In certain preferred embodiments, the electrolyte solution may have a pH in a range of about 7.4 to 10.

Particularly, the kit may include the co-reactant including 4-dimethylaminopyridine at a concentration of greater than 0 mM and about 20 mM or less, greater than 0 mM and about 15 mM or less, greater than 0 mM and about 10 mM or less, or greater than 0 mM and about 7 mM or less.

In certain embodiments, an electrochemiluminescent label including a ruthenium compound may be used to label an antigen or antibody, and electrochemiluminescent immunoassays by immunoreaction and ECL reaction may be performed. For the specific chemiluminescent reaction which is induced by electrochemistry at the surface of an electrode, an antibody (Ab) may be labeled with an electrochemiluminescent reagent ruthenium pyridine and a carrier may be coated with an antigen or antibody which forms a complex with the corresponding antigen or antibody in the sample by a specific mode of immune response.

Label-conjugated complexes may be separated from free labels by a separation technique. The antigen (Ag) or antibody (Ab) may be quantitatively or qualitatively measured by the luminescence intensity of ruthenium pyridine at an electrode. The co-reactant pyridine derivative flows or put into an electrochemical cell and a voltage may be applied to initiate the ECL reaction.

The system or kit for electrochemiluminescence immunoassay or molecular diagnosis according to various exemplary embodiments of the present invention may require significantly reduced amounts of antigens and/or antibodies for detection as applied to a diagnostic device. Because the ECL luminescence intensity emitted from the electrochemical reaction between the pyridine derivative and ruthenium pyridine may be 20 times greater than that between tripropylamine and ruthenium pyridine, much less amount of antigen and antibody can be detected, and therefore, the amount of antibody required for detection can be efficiently reduced. In addition, kits or analytical devices may be relatively inexpensive.

Furthermore, the system or kit for electrochemiluminescence immunoassay or molecular diagnosis as described herein may provide increased or effective detection signals and improved detection sensitivity for the analyte compared to the conventional techniques using tripropylamine. In particular, the pyridine derivative represented by Chemical Formula (1) can co-react with ruthenium pyridine, which is an electrochemiluminescent label, to significantly increase the luminescence intensity. The co-reactant as described herein or the composition including the same can be widely applied to in vitro diagnostic devices such as immunoassay.

### EXAMPLE

Hereinafter, exemplary examples are only provided such that the present invention may be more easily understood, and the content of the present invention is not limited by the following examples.

### Experimental Example: Common Experiment and Measurement Method

Before starting the electrochemiluminescence measurement in the ECL detector of the Example, a cell was washed with a washing solution containing ethanol and water, and dried with N₂ gas. The surface of a working electrode was also polished with an alumina (particle size of about 0.05 μm) slurry, sonicated with a mixture of deionized water (DI) and ethanol (1:1 v/v) within 5 minutes, rinsed and dried with N₂ gas.

After cleaning, the electrochemical cell was filled with an electrolyte solution and attached to a photomultiplier tube (PMT). Then, a voltage was applied between the working electrode and the reference electrode by supplying power, and through this, a liquid sample of the cell was charged with a current predetermined by a control signal, and a reaction was initiated at the working electrode. ECL light generated at the working electrode passed through a photomultiplier tube (PMT), and the ECL light from the ECL response to the working electrode was sensed by an optical detector such as a photomultiplier tube (PMT) located above and adjacent to the electrochemical cell. Further, the main body was completely enclosed in a darkroom environment, not shown in a Markush drawing, which allowed the photomultiplier tube (PMT) to receive the ECL light generated from the cell without external interference.

FIG. 1 is an exemplary electrochemiluminescence system for performing electrochemiluminescence measurements according to an exemplary embodiment of the present invention. For electrochemiluminescence measurement, a potentiostat was used to induce a reaction in an electrochemical cell, and the potentiostat and electrochemiluminescence measurement software may be used to measure the luminescence intensity of electrochemiluminescence.

The performance and influential parameters of a newly discovered co-reactant, 4-DMAP were measured, through experiments and compared its performance with those of the existing co-reactants TPrA and DBAE. The measurement of ECL luminescence was performed by supplying 7 mL of electrolyte to the cell, applying a potential to electrodes, and the intensity of ECL light was recorded using a photomultiplier tube (PMT).

Furthermore, an ECL detection method by potential control was performed by sweeping from 0 V to 1.6 V at a rate of 0.1 V/sec. The above voltage values were assigned between the working electrode (glassy carbon electrode, platinum electrode or gold electrode) and the reference electrode (Ag/AgCl or Ag/Ag⁺). Results are shown in the following Examples.

### Example 1: Confirmation of Electrochemical Behavior of Various Electrochemiluminescence Co-Reactants

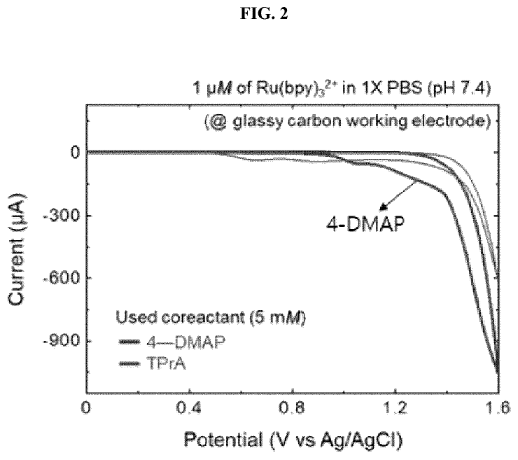

An electrochemiluminescent labeling compound, Ru(bpy)₃²⁺, electrochemiluminescent co-reactants tripropylamine (TPrA), dimethyl ethanolamine (DBAE) and 4-dimethylaminopyridine (4-DMAP), and solvents phosphate buffer solution (PBS) and acetonitrile (ACN) were each provided. These were mixed to prepare each sample, and a cyclic voltammogram (CV) and ECL intensity were measured and recorded for these samples using a potentiostat. A voltage scan started from 0.0 V at a rate of 0.1 V/s. In this case, a glassy carbon electrode, a platinum electrode and a silver electrode were used as working electrodes, and Ag/AgCl or Ag/AgNO₃ was used as a reference electrode. The upper voltage limit was 1.6 V, the lower voltage limit was 0 V, and the final voltage was 0 V. The electrochemical behavior of various electrochemiluminescence co-reactants was measured and shown in FIGS. 2 to 5.

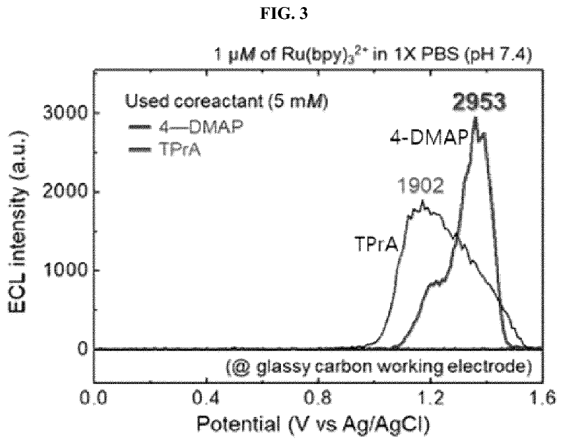

FIGS. 2 and 3 show the results of measuring the cyclic voltammogram (CV) and ECL intensity for the electrochemiluminescence system of Ru(bpy)₃²⁺/TPrA and Ru(bpy)₃²⁺/4-DMAP using glassy carbon materials as working electrodes during periodic potential scanning from 0 V to 1.6 V in PBS, respectively. As a result of FIGS. 2 and 3, the ECL intensity of Ru(bpy)₃²⁺/4-DMAP was greater than that of Ru(bpy)₃²⁺/TPrA.

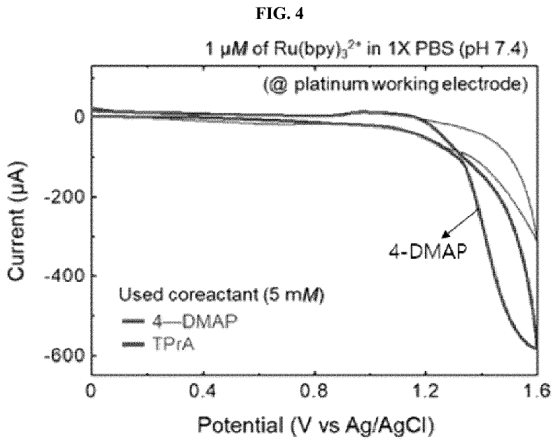

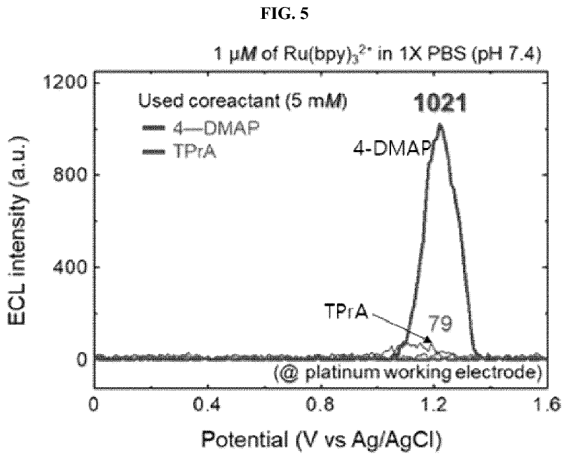

Meanwhile, FIGS. 4 and 5 show the results of measuring the cyclic voltammogram (CV) and ECL intensity for the electrochemiluminescence system of Ru(bpy)₃²⁺/TPrA and Ru(bpy)₃²⁺/4-DMAP using platinum materials as working electrodes during periodic potential scanning from 0 V to 1.6 V in PBS, respectively.

As a result of FIGS. 4 and 5, the anodic current of Ru(bpy)₃²⁺/4-DMAP was greater than that of Ru(bpy)₃²⁺/TPrA. Further, the ECL intensity of Ru(bpy)₃²⁺/4-DMAP was substantially greater than that of Ru(bpy)₃²⁺/TPrA.

Through these results, the platinum electrode was the most favorable working electrode for 4-DMAP oxidation in phosphate buffer saline (PBS), followed by the glassy carbon electrode.

### Example 2: ECL Characteristics According to Concentration of Electrochemiluminescence Co-Reactant

Electrolyte solutions were prepared by varying the concentration of 4-DMAP as an electrochemiluminescence co-reactant, while maintaining the Ru(bpy)₃²⁺ concentration of the sample in PBS constant. Voltage scans were performed using the same procedure described in Example 1, and electrochemiluminescence intensities were measured in triplicate readings for each concentration of the electrochemiluminescence co-reactant.

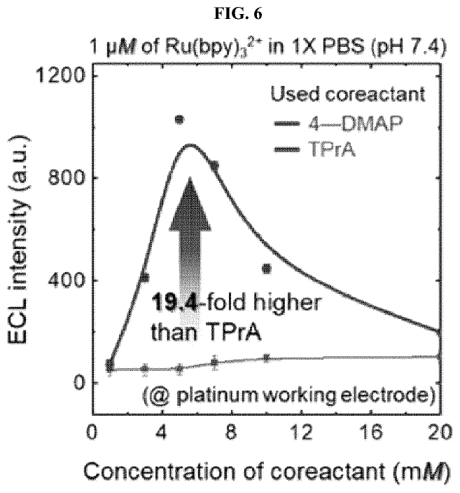

FIG. 6 shows the results of measuring the ECL intensity according to the concentration of 4-DMAP using a platinum electrode as a working electrode. The ECL intensity increased dramatically when 4-DMAP was used in a concentration range of greater than 0 to 5 mM or less. However, the optimal concentration was 5 mM or less because the ECL intensity decreased when the 4-DMAP concentration was higher than 5 mM. Meanwhile, in the case of TPrA, the ECL intensity continued to increase when the TPrA concentration increased.

Through these results, when 4-DMAP was used as an electrochemiluminescence co-reactant, the concentration range of greater than 0 mM and 5 mM or less provided the best luminescence intensity.

### Example 3: ECL Characteristics of Electrochemiluminescence Co-Reactants According to Various Ru(bpy)₃²⁺ Concentrations

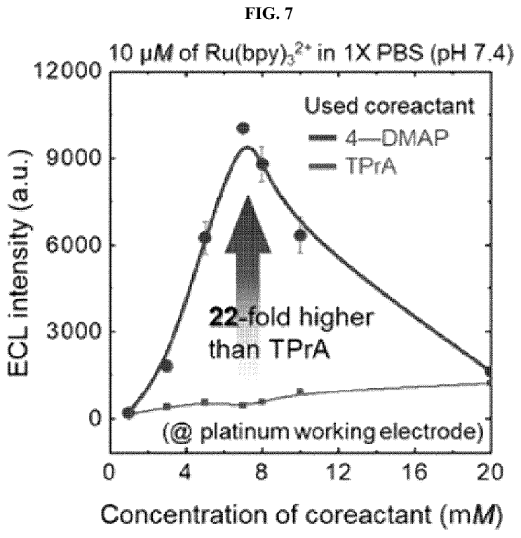

Samples of PBS including various concentrations of Ru(bpy)₃²⁺ were prepared. For voltage scans, the same procedure as described in Example 1 was used. The electrochemiluminescence intensity at each concentration of Ru(bpy)₃²⁺ was measured, and the results are shown in FIG. 7. As a result of FIG. 7, the intensity showed the highest intensity values during ECL measurements using a platinum (Pt) electrode as the working electrode at 10 Ru(bpy)₃²⁺ and a 7 mM 4-DMAP concentration, and the intensity decreased when the 4-DMAP concentration exceeded 7 mM.

Overall, the optimum concentration of 4-DMAP used as a co-reactant in a PBS solution including 10 μM Ru(bpy)₃²⁺ was around 7 mM.

### Example 4: ECL Characteristics of Electrochemiluminescence Co-Reactant According to pH

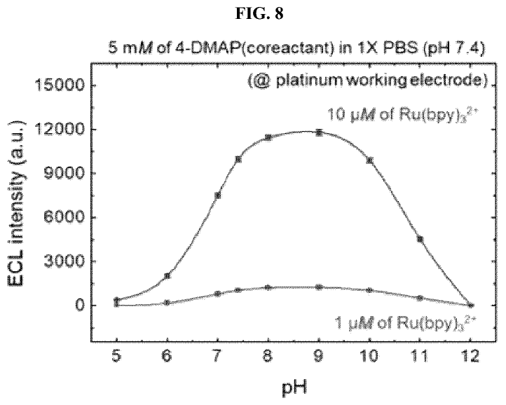

The ECL characteristics of the electrochemiluminescence co-reactant at various pH values were investigated. Electrochemiluminescence from the electrochemiluminescence co-reactant was measured for PBS samples including constant concentrations of Ru(bpy)₃²⁺, except that the pH was varied from 5 to 12. Voltage scans were performed using the same procedure as described in Example 1. The electrochemiluminescence intensity was measured three times for different pH values, and the results are shown in FIG. 8.

FIG. 8 shows the results of examining 4-DMAP performance at different pH values, and a pH range from 7.4 to 10 may be the most optimal range for 4-DMAP performance when 1 IM Ru(bpy)₃²⁺ and 5 mM 4-DMAP were used. Similarly, when 10 μM Ru(bpy)₃²⁺ and 7 mM 4-DMAP were used, the ECL intensity was inferior at a pH value of less than 7.4 and 10 or greater.

### Example 5: ECL Characteristics of Electrochemiluminescence Co-Reactant Under Various Solvents

Samples were prepared using an acetonitrile (ACN) solution instead of a PBS solution.

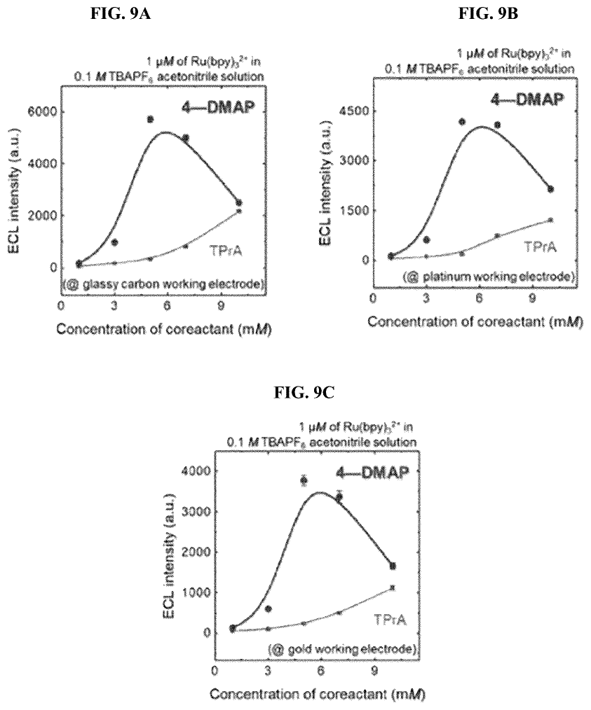

FIGS. 9A-9C shows the results of measuring the electrochemiluminescence intensity using an acetonitrile (ACN) solution instead of PBS. When a glassy carbon electrode (FIG. 9A), a platinum electrode (FIG. 9B) and a gold electrode (FIG. 9C) were each used as a working electrode, the ECL intensities of Ru(bpy)₃²⁺/4-DMAP at relatively low concentrations of 3 mM, 5 mM and 7 mM were greater than those of Ru(bpy)₃²⁺ TPrA.

### Example 6: ECL Characteristics of Electrochemiluminescence Co-Reactant According to Various Luminescence Materials

Each sample was prepared by mixing, as electrochemiluminescent labels, iridium-based transition metal complex compounds iridium bis[5-(trifluoromethyl)-2-(4-(trifluoromethyl)phenyl)pyridine] picolinate ((Ir(ppy-(CF₃)₂)₂(pico)), bis(2-phenylpyridine)(4,4′-di-tert-butyl-2,2′-dipyridyl)iridium(III) (hexafluorophosphate) (Ir(dtbbpy)(ppy)₂PF₆) and Tris[2-(p-tolyl)pyridine]iridium(III) (Ir(mppy)₃), 4-dimethylaminopyridine (4-DMAP), and a supporting electrolyte tetrabutylammonium hexafluorophosphate (TBAPF₆) with acetonitrile. Since the luminescence characteristics of these samples are very sensitively affected by oxygen and moisture, measurements were performed under a glove box in a nitrogen environment.

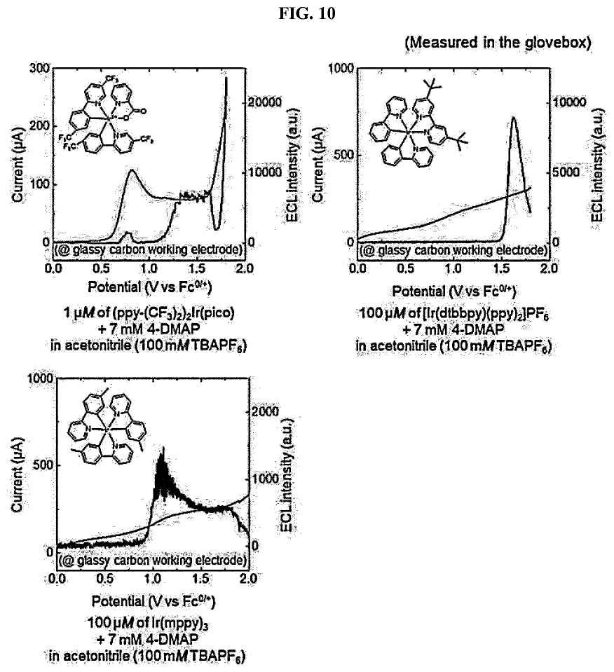

A linear sweep voltammogram (LSV) and ECL were measured and recorded for these samples. The scan rate started from 0.0 V at a rate of 0.1 V/s, and samples were scanned in a voltage range within 2.0 V. In this case, a glassy carbon electrode was used as a working electrode, Ag/Ag⁺ (3M AgNO₃) was used as a reference electrode, and Pt was used as an auxiliary electrode, and the results are shown in FIG. 10.

FIG. 10 shows the results of showing a linear sweep voltammogram and electrochemiluminescence intensity when 4-DMAP as a co-reactant and an iridium-based transition metal complex compound were used in an acetonitrile (ACN) solution, and glassy carbon was used as a working electrode. As a result of FIG. 10, even when the iridium-based transition metal complex compound was used as an electrochemiluminescent label, the ECL intensity was shown to be high, and the anodic current was also high.

Through these results, it is possible to use various types of transition metal complex compounds as electrochemiluminescent labels.

### Example 7: Comparison of ECL Detection Curves According to Concentration of Ru(bpy)32+ Using Electrochemiluminescence Co-Reactant

A PBS solution including a co-reactant at a concentration of 7 mM was prepared, and electrochemiluminescence signals generated when the concentration of Ru(bpy)₃²⁺ was changed were observed. Voltage scans were taken using the same procedure as described in Example 1. Electrochemiluminescence intensity was measured in triplicate readings under each condition.

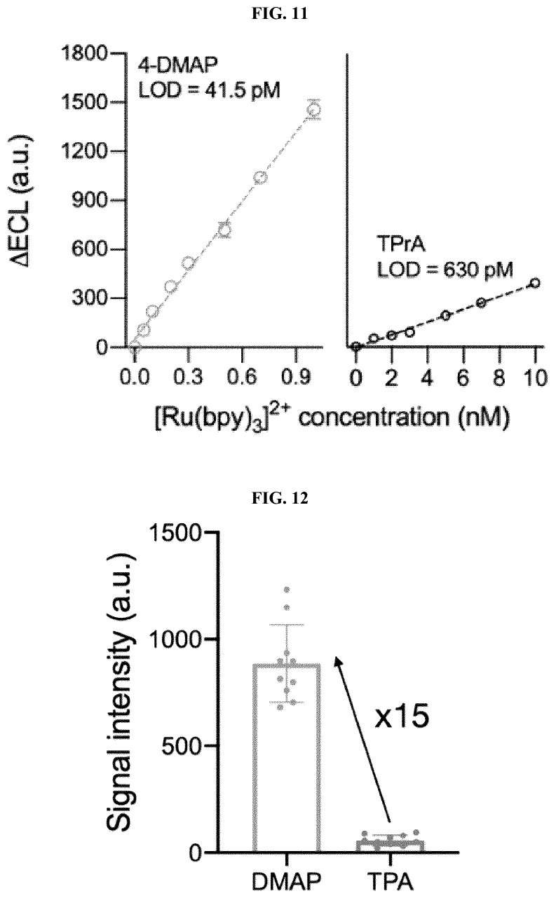

FIG. 11 shows the electrochemiluminescence intensity generated according to the concentration of Ru(bpy)₃²⁺ in each solution using 4-DMAP and tripropylamine as co-reactants. In the case of the tripropylamine co-reactant (right, black graph), electrochemiluminescence detection signals can be obtained for Ru(bpy)₃²⁺ in a concentration range of 0 to 10 nM, and the limit of detection for Ru(bpy)₃²⁺ was shown to be 0.63 nM (630 μM). In contrast, 4-DMAP shows a high electrochemiluminescence signal for Ru(bpy)₃²⁺ at a concentration of 0 to 0.1 nM, and the limit of detection was 0.0415 nM (41.5 μM), which shows a detection sensitivity at least 15 times better than tripropylamine.

Through these results, an electrochemiluminescence signal could be provided for the luminophore at a lower concentration than the concentration of tripropylamine, which can provide better detection sensitivity when used for immunodiagnosis or molecular diagnosis.

### Example 8: Comparison of ECL Detection Curves According to Concentration of Ru(bpy)₃²⁺ Using Electrochemiluminescence Co-Reactant

Chemiluminescent immunodiagnosis was performed on a “severe acute respiratory syndrome (SARS-CoV-2) neutralizing antibody (anti-SARS-CoV-2)” present in human saliva using 4-DMAP and tripropylamine as co-reactants, respectively. Saliva samples of ten vaccinated persons were collected and a total of three steps of electrochemical immunoassays were performed.

30 μL of supernatant of centrifuged human saliva was mixed with a magnetic bead (2 μm diameter) reagent (capture reagent) to which a pre-prepared SARS-CoV-2 antigen was attached. The neutralizing antibody present in human saliva in this step formed a sandwich immunoconjugate with an antigen previously immobilized on magnetic beads. After incubating this mixture for 30 minutes (37° C.), only magnetic beads were collected and washed twice with PBS. Then, the beads were mixed with 30 μL of a 5 μg/mL human IgG antibody reagent (ECL signal generating reagent) to which a Ru(bpy)₃²⁺ label is attached. Finally, only the magnetic beads were collected from the mixture above and captured on gold-printed electrodes. Electrochemiluminescence signals were measured by dropping 7 mM MDMAP or a 7 mM tripropylamine buffer solution (PBS) on the magnetic beads on the surface of the electrode, and applying a voltage to each.

FIG. 12 shows the intensities of the electrochemiluminescence signals measured when 4-DMAP was used as a co-reactant and tripropylamine was used as a co-reactant in electrochemical immunodiagnosis for anti-SARS-CoV-2 neutralizing antibodies using saliva samples from 10 persons inoculated with a vaccine. As a result, when 4-DMAP was used as a co-reactant for the same 10 saliva samples, detection signals appeared to be at least 15 times greater than the signals received using tripropylamine.

As results, substantially increased or improved detection sensitivity could be obtained when 4-DMAP was used as a co-reactant in electrochemiluminescence immunodiagnosis or molecular diagnosis.

Therefore, the luminescence intensity between the pyridine derivative represented by Chemical Formula (I) as described herein and ruthenium pyridine was at least 20-fold that obtained using tripropylamine, indicating that luminescence sensitivity was excellent. Therefore, since the pyridine derivative represented by Chemical Formula (1) as described herein can replace tripropylamine which has been conventionally used as a co-reactant and the voltage for light emission can be controlled to improve luminescence efficiency, wide application potential in various bioanalyses such as immunoassays can be provided.

Although exemplary embodiments of the present invention have been described in detail, it is obvious to those skilled in the art that such specific descriptions are only exemplary embodiments and the scope of the present invention is not limited thereby. Accordingly, the substantial scope of the present invention will be defined by the appended claims and equivalents thereof.

What is claimed:

1. An electrolyte composition comprising:
an electrochemiluminescent label;
an electrochemiluminescence co-reactant comprising a compound having a structure of Chemical Formula (I): 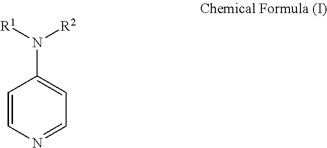
wherein R¹ and R² are the same as or different from each other, and each R¹ and R² is independently a hydrogen, a halogen, a C₁-C₆ straight-chain alkyl group, a C₁-C₆ branched-chain alkyl group, or C₃-C₆ cyclic alkyl group, a C₁-C₆ alkoxy group; and a C₁-C₆ haloalkyl group, or a pharmaceutically acceptable salt thereof.

2. The electrolyte composition of claim 1, wherein each R¹ and R² is independently a C₁-C₄ straight-chain alkyl group, a C₁-C₄ branched-chain alkyl group, or a C₁-C₄ haloalkyl group.

3. The electrolyte composition of claim 1, wherein the electrochemiluminescence co-reactant comprises 4-dimethylaminopyridine (4-DMAP).

4. The electrolyte composition of claim 1, further comprising a solvent component comprising one or more selected from the group consisting of water, phosphate buffer saline (PBS), acetonitrile (ACN), dichloromethane, ethanol, methanol, tetrahydrofuran (THF), dimethyl sulfoxide (DMSO), dimethylformamide (DMF), ethylene carbonate (EC) and propylene carbonate (PC).

5. The electrolyte composition of claim 4, wherein the electrolyte composition comprises 4-dimethylaminopyridine at a concentration of greater than 0 mM and about 20 mM or less.

6. The electrolyte composition of claim 5, wherein the electrolyte composition has a pH in a range of about 5 to 12.

7. The electrolyte composition of claim 1, wherein the electrochemiluminescent label comprises one or more selected from the group consisting of a transition metal complex compound, a luminescent organic semiconductor, a quantum dot material, perovskite nanoparticles, metal nanoparticles and carbon nanoparticles.

8. An electrochemiluminescence system comprising:
an electrochemical cell;
an electrolyte solution comprising a compound having a structure of Chemical Formula (I): 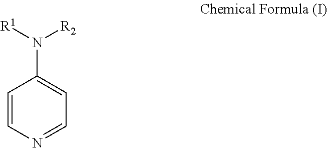
wherein R¹ and R² are the same as or different from each other, and each R¹ and R² is independently a hydrogen, a halogen, a C₁-C₆ straight-chain alkyl group, a C₁-C₆ branched-chain alkyl group, or C₃-C₆ cyclic alkyl group, a C₁-C₆ alkoxy group; and a C₁-C₆ haloalkyl group, or a pharmaceutically acceptable salt thereof; and
a photodetector connected to the electrochemical cell.

9. The electrochemiluminescence system of claim 8, wherein the electrochemical cell comprises a working electrode comprising one or more selected from the group consisting of carbon, platinum (Pt), gold (Au), silver (Ag), nickel (Ni), stainless steel, palladium, tin, indium and silicon elements.

10. The electrochemiluminescence system of claim 8, wherein each R¹ and R² is independently a C₁-C₄ straight-chain alkyl group, a C₁-C₄ branched-chain alkyl group, or a C₁-C₄ haloalkyl group.

11. The electrochemiluminescence system of claim 8, wherein the electrochemiluminescence co-reactant comprises 4-dimethylaminopyridine (4-DMAP).

12. The electrochemiluminescence system of claim 11, wherein the electrolyte solution comprises 4-dimethylaminopyridine at a concentration of greater than 0 mM and about 20 mM or less.

13. The electrochemiluminescence system of claim 8, wherein the electrochemiluminescent label comprises one or more selected from the group consisting of a transition metal complex compound, a luminescent organic semiconductor, a quantum dot material, perovskite nanoparticles, metal nanoparticles and carbon nanoparticles.

14. The electrochemiluminescence system of claim 8, wherein the electrolyte solution comprises a solvent component comprising one or more selected from the group consisting of phosphate buffer saline (PBS), acetonitrile (ACN), dichloromethane, ethanol, methanol, tetrahydrofuran (THF), dimethyl sulfoxide (DMSO), dimethylformamide (DMF), ethylene carbonate (EC) and propylene carbonate (PC).

15. The electrochemiluminescence system of claim 8, wherein the electrolyte solution has a pH in a range of about 5 to 12.

16. A kit for electrochemiluminescence immunoassay or molecular diagnosis, comprising an electrolyte composition of claim 1.

17. A method of detecting a biological material in a sample, comprising:
(a) placing an admixture comprising an electrolyte solution of claim 1 and a sample into an electrochemical cell; and
(b) measuring an electrochemiluminescence intensity (ECL intensity) from the admixture while varying an input voltage.

18. The method of claim 17, wherein the biological material comprises a peptide, a protein, an antibody, an antigen, a nucleic acid, a virus, a cell, a tissue, or combinations thereof.

19. The method of claim 17, wherein the electrochemiluminescence intensity (ECL intensity) is detected as a photodetector.

20. The method of claim 17, further comprising calculating a concentration of the biological material in the sample, or determining the presence of the biological material in the sample.
